(19) United States
(12) Patent Application Publication
(10) Pub. No.: US 2022/0304565 A1
(43) Pub. Date: Sep. 29, 2022

(54) VAGINAL PLUG FOR VAGINOSCOPY AND FACILITATING ATRAUMATIC TRANSCERVICAL INSERTION OF A HYSTEROSCOPE

(71) Applicants: Fauzia Akbary, Detroit, MI (US); Maurice Andre Recanati, New York, NY (US); Katherine Kramer, Philadelphia, PA (US)

(72) Inventors: Fauzia Akbary, Detroit, MI (US); Maurice Andre Recanati, New York, NY (US); Katherine Kramer, Philadelphia, PA (US)

(21) Appl. No.: 17/703,143

(22) Filed: Mar. 24, 2022

Related U.S. Application Data
(60) Provisional application No. 63/165,365, filed on Mar. 24, 2021.

(51) Int. Cl. A61B 1/32; A61B 1/303; A61B 17/42; A61B 1/018

(52) U.S. Cl. CPC A61B 1/32; A61B 1/303; A61B 17/42; A61B 1/018

(57) ABSTRACT

A medical device for use during a gynecological procedure includes a main body, an attachment rim disposed at a first end of the main body, and a valve securing element disposed at a second end of the main body. A valve is disposed between the main body and the valve securing element.

### CROSS-REFERENCE TO RELATED APPLICATIONS

This application claims the benefit of U.S. Provisional Application Ser. No. 63/165,365, filed on Mar. 24, 2021. The entire disclosure of the above application is incorporated herein by reference.

### FIELD

The present invention relates generally to a medical device and, more particularly, to a medical device used during a gynecological procedure.

### INTRODUCTION

This section provides background information related to the present disclosure which is not necessarily prior art.

It is common for women to undergo gynecological examinations and procedures leading up to and throughout adulthood. Medical professionals use various medical equipment and devices in order to view the female reproductive track including the vulva, vagina, cervix, uterus, and fallopian tubes. For example, a hysteroscope may be used to aid a physician in visualizing the uterine cavity, collecting tissue samples for further analysis, and performing certain gynecological procedures.

Presently known medical devices that are used in the gynecological field have a number of drawbacks and limitations. As an example, many known hysteroscopes and methods of using hysteroscopes require insertion of a speculum to locate a patient's cervix followed by insertion of a tenaculum to grasp the cervix. This can result in bleeding, trauma to the cervix, and other injuries such as uterine perforations. Additionally, patients may experience pain and discomfort. Unfavorably, known hysteroscopes do not allow a physician or medical professional to visualize the vaginal mucosa, cervix, and other areas of the female reproductive tract, especially in younger patients, patients of reproductive age, multiparous or obese patients, and patients with herniating pelvic organs. Additionally, known hysteroscopes do not prevent the leaking of fluids from the patient, in operation.

In one particular example, U.S. Pat. No. 10,842,371 teaches a vaginal speculum that allows for insertion of instruments into a patient's vagina. However, the device does not allow for vaginoscopy and does not facilitate transcervical insertion of a hysteroscope. In another example, U.S. Pat. No. 11,147,444 teaches a speculum that allows for minimal visualization of the female reproductive system by the physician. However, the device does not prevent leakage of fluids from the patient during the examination or procedure.

Accordingly, there is a continuing need for a medical device that is capable of being inserted into a patient comfortably and atraumatically, that is adaptable amongst patients having variable anatomy, that allows a physician to easily visualize the reproductive system of the patient and perform a complete gynecological exam, even in patients that may have complicating factors, and that prevents the leakage of fluids from the patient during examination. Desirably, the medical device would be easy to use, inexpensive to manufacture, and configured to work in combination with a variety of medical devices.

### SUMMARY

In concordance with the instant disclosure, a medical device that is capable of being inserted into a patient comfortably and atraumatically, that is adaptable amongst patients having variable anatomy, that allows a physician to easily visualize the reproductive system of the patient and perform a complete gynecological exam, even in patients that may have complicating factors, that prevents the leakage of fluids from the patient during examination, that is easy to use and inexpensive to manufacture, and that is configured to work in combination with a variety of medical devices, has surprisingly been discovered.

In certain embodiments, a medical device for use during a gynecological procedure includes a main body, an attachment rim disposed at a first end of the main body, and a valve securing element disposed at a second end of the main body. A valve is disposed between the main body and the valve securing element.

In certain embodiments, a medical device for use during a gynecological procedure includes a main body, an attachment rim disposed at a first end of the main body, and a valve securing element disposed at a second end of the main body. A first valve is disposed between the main body and the valve securing element, and a second valve is disposed between the main body and the valve securing element.

In certain embodiments, a method of using a medical device during a gynecological procedure comprises a step of providing a medical device. The medical device includes a main body, an attachment rim disposed at a first end of the main body, a valve securing element disposed at a second end of the main body, and a valve disposed between the main body and the valve securing element. The method can further include one or more of deforming the medical device, inserting the medical device into a patient, releasing the medical device in the patient, performing a gynecological procedure, deforming the medical device, and removing the medical device from the patient.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a top perspective view of a medical device positioned with respect to a schematic of a female reproductive tract, according to one embodiment of the present disclosure;

FIG. 2 is a top perspective view of the medical device of FIG. 1;

FIG. 3 is an exploded top perspective view of the medical device of FIG. 1;

FIG. 4 is a side elevational view of the medical device of FIG. 1;

FIG. 5 is a top plan view of the medical device of FIG. 1;

FIG. 6 a bottom perspective view of a medical device according to another embodiment of the present disclosure;

FIG. 7 is a top perspective view of a medical device positioned with respect to a schematic of a female reproductive tract according to another embodiment of the present enclosure; and

FIG. 8 is a method of using a medical device according to one embodiment of the present disclosure.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed, unless expressly stated otherwise. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

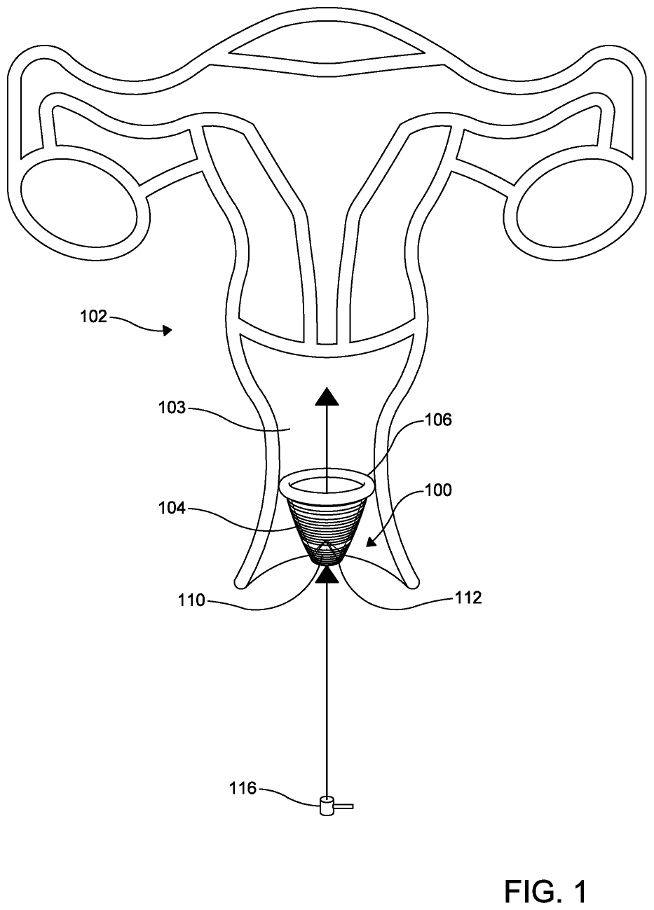

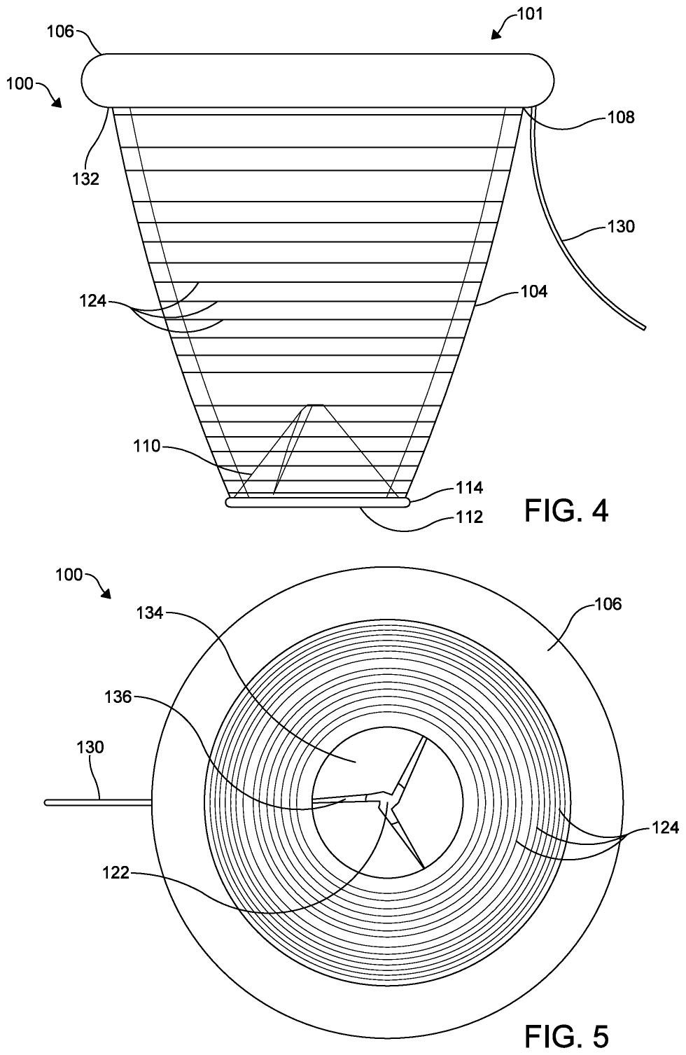

With reference to FIGS. 1-5, a medical device 100 for use during a gynecological examination or procedure is shown. The medical device 100 may be positioned in a female reproductive tract 102 of a patient by a medical professional and may include a main body 104, an attachment rim 106 disposed at a first end 108 of the main body 104, a valve 110, and a valve securing element 112 configured to secure the valve 110 to a second end 114 of the main body 104. The medical device 100 may have a predetermined shape 101, as shown in FIGS. 1-5.

The medical device 100 may be used during diagnostic and operative gynecological examinations and procedures in combination with one or more additional examination tools 116 such as a hysteroscope, as one non-limiting example. The medical device 100 may be fabricated using any biosafe, hypoallergenic material or combination of materials suitable for placement within a human body. As non-limiting examples, any combination of biosafe plastic, rubber, silicon, polycarbonate, and metal may be used. According to certain embodiments, each of the main body 104, the attachment rim 106, the valve 110, and the valve securing element 112, may be integral with one another, or may be separate components permanently or removably connected to one another. According to certain more particular embodiments, the medical device 100 may be devoid of sharp angles and edges, and may not include removable, separable, and loose parts and components.

A skilled artisan may select any suitable shape and size for the medical device 100. The medical device 100 may be configured to be completely or partially retained in or along the female reproductive tract 102 of the patient. As shown in FIG. 1, in certain embodiments, the medical device 100 may be entirely disposed in a vaginal cavity 103 of the patient during examination, as one non-limiting example. According to certain other embodiments, the medical device 100 may be partially disposed in the vaginal cavity 103 or other area of the female reproductive tract 102 such that one of the first and second ends 108, 114 of the main body 104 may protrude from the patient.

The main body 104 of the medical device 100, according to certain embodiments, may be conically-shaped, bell-shaped, or cup-shaped, as non-limiting examples, and may define a hollow cavity 118. A skilled artisan may select any suitable shape for the main body 104 of the medical device 100 capable of being inserted into and comfortably retained in the female reproductive tract 102, as desired. A first opening 120 may be disposed at the first end 108 of the main body 104, and a second opening 122 may be disposed at the second end 114 of the main body 104, according to certain embodiments.

The main body 104 of the medical device 100 may be fabricated using a flexible, pliable, moldable material or combination of materials. The main body 104 may be capable of being deformed during insertion and removal of the medical device 100. The main body 104 of the medical device 100 may include elastomeric and shape-memory properties configured to revert the medical device 100 to the predetermined shape 101 after deformation and insertion or removal of the medical device 100, according to certain embodiments. The main body 104, according to certain embodiments, may be fabricated using a strong, resilient material such as plastic or polycarbonate, as non-limiting examples, that is capable of withstanding pressure from fluid and retracting vaginal walls of the patient after insertion and during the gynecological examination. In certain embodiments, an elastomeric material such as an organosilicon oxide polymer or a medical grade silicone rubber, as non-limiting examples, may be used, as determined by one of skill in the art.

In certain more particular embodiments, the main body 104 may be fabricated using a transparent material such a transparent PVC plastic, transparent polycarbonate, or transparent silicone rubber, as non-limiting examples. A skilled artisan may select any suitable material having sufficient strength and flexibility for the main body 104 of the medical device 100. The main body 104 may include grooves 124, ridges, or other surface texture disposed on or in an outer surface 126 of the main body 104 of the medical device 100 to enhance gripping of the medical device 100 by the medical professional during insertion and removal of the medical device 100. The grooves 124 may be integral with the main body 104 or separate components permanently affixed to the main body 104, as determined by one of skill in the art. Any number and configuration of grooves 124 or surface texture may be employed, as desired.

The attachment rim 106 may be disposed around the first end 108 of the main body 104 of the medical device 100. The attachment rim 106 may be adapted to form a fluid tight seal between the outer surface 126 of the attachment rim 106 and the vaginal wall or another area of the patient's female reproductive tract 102. Accordingly, in certain embodiments, the attachment rim 106 is configured to have a maximized outer surface area 128 that is in contact with the desired area of the female reproductive tract 102 once the medical device 100 is properly positioned. The maximized outer surface area 128 of the attachment rim 106 may be smooth, according to certain embodiments, and use a tension or friction fit to remain securely positioned in the female reproductive tract 102. Alternatively, the maximized outer surface area 128 may include a surface texture configured to grip the desired area of the female reproductive tract 102 and use a combination of surface texture, traction, and tension or friction fit to remain securely positioned in the female reproductive tract 102.

According to certain embodiments, the attachment rim 106 may be generally cylindrical or ring-shaped, and may extend around the first opening 120 at the first end 108 of the main body 104. The attachment rim 106 may be flexible, according to certain embodiments, and may be made from any biosafe, hypoallergenic material such as silicone, rubber, or plastic, as non-limiting examples. Any shape, size, and configuration adapted to optimally secure the medical device 100 against the vaginal wall or other area of the female reproductive tract 102 may be used, as desired.

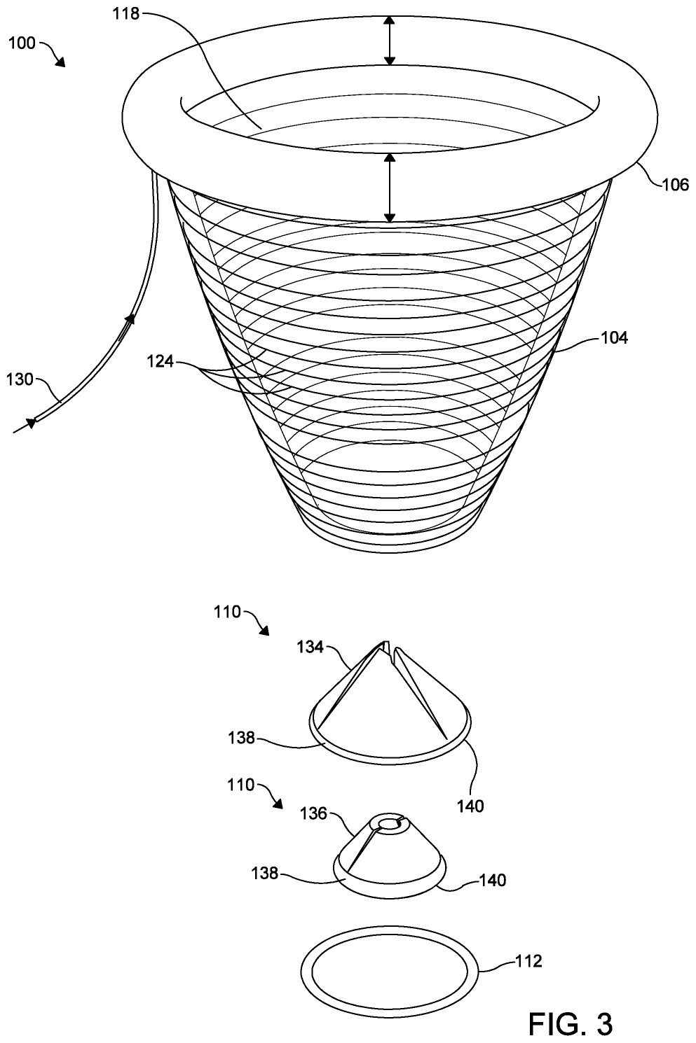

In a more particular embodiment, as shown in FIGS. 1-5, the attachment rim 106 may be fabricated using an expandable or inflatable plastic, rubber, or silicon, as non-limiting examples. The attachment rim 106 may be substantially hollow and may be in fluid communication with a tube 130. With reference to FIG. 3, upon insertion of the medical device 100, the medical professional may introduce a desired quantity of fluid (e.g., air) through the tube 130 and into the attachment rim 106 such that a desired tension between the vaginal wall or other desired area of the patient's female reproductive tract 102 and the attachment rim 106 is obtained. Likewise, the medical professional may use the tube 130 to remove fluid from the attachment rim 106 prior to removing the medical device 100 from the patient.

In certain embodiments, the tube 130 may extend downwardly from a bottom surface 132 of the attachment rim 106 or any area of the attachment rim 106 that is not in direct contact with the vaginal wall or other area of the female reproductive tract 102. In certain embodiments, the tube 130 may extend downwardly from a recessed area (not shown) of the attachment rim 106 so that the tube 130 does not irritate or injure the female reproductive tract 102 during insertion or removal of the medical device 100.

In another more particular embodiment, the attachment rim 106 may have a ring-shaped configuration capable of being deformed or folded during insertion and removal of the medical device 100. As non-limiting examples, biosafe shape memory natural rubber or memory foam may be used. A skilled artisan may select any material or combination of materials that include any suitable elastic or shape-memory properties capable of reverting the attachment rim 106 back to the predetermined shape 101 after insertion or removal of the medical device 100. The attachment rim 106 may be fabricated using a strong, resilient material that is capable of withstanding pressure from fluid and the retracting vaginal walls of the patient after insertion and during the gynecological examination.

In yet another more particular embodiment, the attachment rim 106 may include a hollow, ring-shaped configuration (not shown). The attachment rim 106 may include elastic or shape-memory properties capable of reverting the attachment rim 106 back to the predetermined shape 101 after insertion or removal of the medical device 100. A string (not shown) may extend through the hollow, ring-shaped attachment rim 106. At least one end of the string may extend outwardly from a bottom surface of the attachment rim 106. The end of the string may be accessible to the medical professional. The medical professional, by way of pulling on the end of the string, may use the string to deform or collapse the attachment rim 106 during insertion and removal of the medical device 100.

A skilled artisan may employ any attachment rim 106 capable of exerting tension or pressure on the vaginal wall or other desired area of the patient's female reproductive tract 102 and forming a fluid tight seal. The attachment rim 106 may be further capable of being deformed, folded, or otherwise collapsed or manipulated to allow for easy insertion and removal of the medical device 100. Any suitable material, combination of materials, and configuration may be used to form the attachment rim 106, as desired.

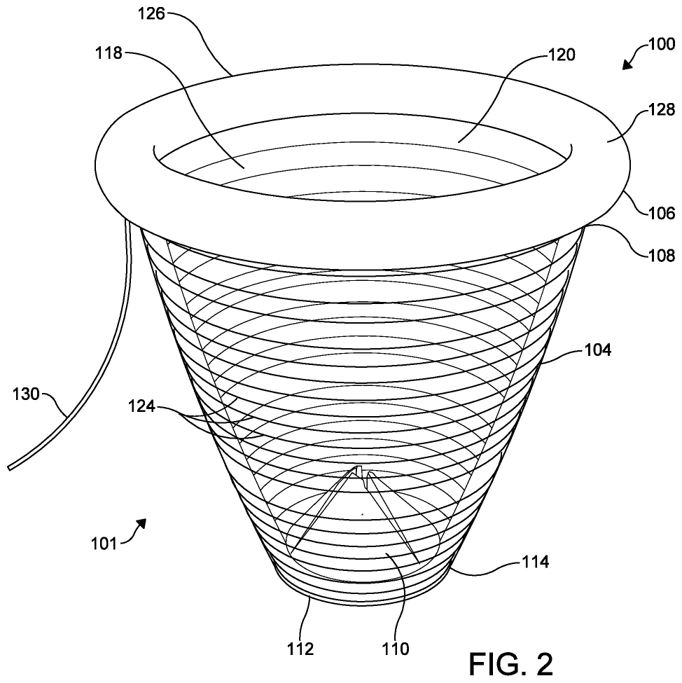

The valve 110, according to certain embodiments, may be a one-way valve 110 positioned in the second opening 122 at the second end 114 of the main body 104, as shown in FIGS. 2-5. The valve 110 may be fabricated using any suitable biosafe, hypoallergenic material, such as silicon or rubber, as non-limiting examples. The valve 110 may be fabricated using a flexible, strong material adapted to receive an examination tool 116 and withstand pressure from water or other fluids during a gynecological procedure, according to certain embodiments. A skilled artisan may select any suitable valve 110 having any configuration and including any desired material or combination of materials, as desired.

According to certain embodiments, the valve 110 may be configured to allow insertion of the hysteroscope or other examination tool 116 during a gynecological examination or procedure while preventing fluid leakage from the patient. More specifically, the valve 110 may prevent distention fluid from leaking out of the female reproductive tract 102 during a hysteroscopy or other gynecological procedure. The valve 110, according to certain embodiments, may be adapted to accommodate hysteroscopes and other examination tools 116 having various diameters. As one non-limiting example, a flap valve 110 may be used such that examination tools 116 having variable diameters may be inserted through the valve 110. Any suitable valve 110 configured to prevent fluid from leaking out from the patient and receive a hysteroscope or other examination tool 116 may be used. A duckbill valve or gel-port, as additional non-limiting examples, may also be used, as determined by one of skill in the art.

According to certain embodiments, with renewed reference to FIGS. 2-5, more than one valve 110 may be disposed in the second opening 122 at the second end 114 of the main body 104. In certain more particular embodiments, as one non-limiting example, a first valve 134 and a second valve 136 may be disposed in the second opening 122 of the second end 114 of the main body 104. In a more particular embodiment, the first valve 134 may be a tri-leaflet valve, as shown in FIGS. 1-5, and the second valve 136 may be a bi-leaflet or duckbill valve, as shown in FIG. 3. The first valve 134 and the second valve 136 may be configured to prevent pressurized water or other fluids from leaking from the patient while simultaneously accepting hysteroscopes or other examination tools 116 having variable diameters during a gynecological procedure.

The valve 110 may be secured to the main body 104 of the medical device 100 using the valve securing element 112. The valve securing element 112 may be generally cylindrical or ring-shaped, and may extend around the second opening 122 at the second end 114 of the main body 104. The valve securing element 112 may be rigid or semi-rigid, according to certain embodiments, and may be made from any biosafe, hypoallergenic material such as plastic or metal, as non-limiting examples. Any shape, size, and configuration adapted to securely connect the valve 110 to the main body 104 of the medical device 100 and militate against deformation of the valve 110 during insertion and removal of the medical device 100 may be used, as desired. The valve 110 may be integral with one or both of the main body 104 and the valve securing element 112, or may be permanently connected or fastened to one or both of the valve securing element 112 and the main body 104 of the medical device 100. Chemical adhesives, vulcanization, and ultrasonic welding, as non-limiting examples, may be used to permanently fasten the valve 110 to one or both of the valve securing element 112 and the main body 104 of the medical device 100, as determined by one of skill in the art. In certain embodiments including the first valve 134 and the second valve 136, each of the first and second valves 134, 136 may be separately fastened to valve securing element 112 independent from one another.

According to certain more particular embodiments, as shown in FIG. 3, the valve 110 may include a lip 138 extending around a base 140 of the valve 110. The lip 138 may be secured between the second end 114 of the main body 104 and the valve securing element 112. A skilled artisan may use any appropriate means for securing the valve 110 between the valve securing element 112 and the main body 104 of the medical device 100, as desired.

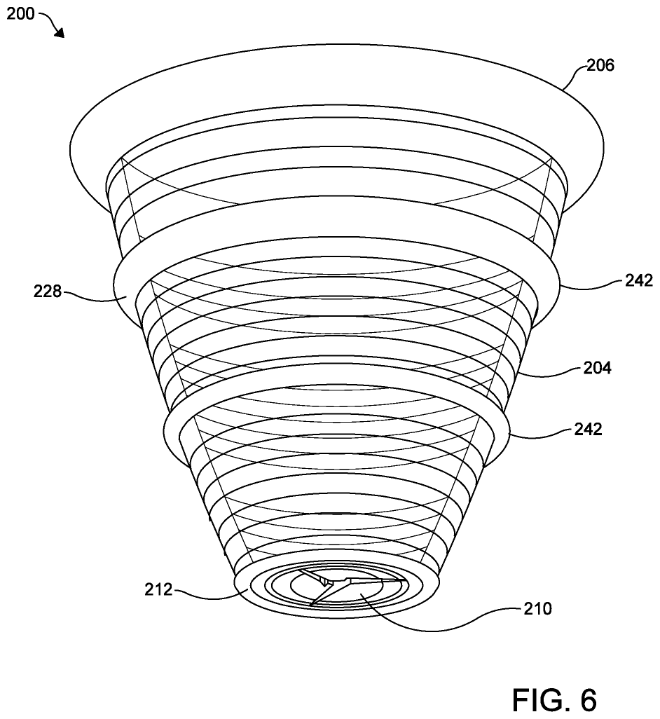

With reference to FIG. 6, a medical device 200 including a main body 204, an attachment rim 206, a valve 210, and a valve securing element 212, according to another embodiment, is shown. The main body 204 of the medical device 200 may include one or more cylindrical support rings 242 disposed in or around the main body 204 of the medical device 200, according to certain embodiments. The support rings 242 may have a flexible, predetermined ring-shaped configuration capable of deformation during insertion and removal of the medical device 200. The support rings 242 may be adapted to improve the fluid tight seal between the medical device 200 and the vaginal walls or other area of the patient's female reproductive tract. Accordingly, in certain embodiments, the support rings 242 are configured to have a maximized outer surface area 228 that is in contact with the desired area of the female reproductive tract once the medical device 200 is properly positioned. The maximized outer surface area 228 of the support rings 242 may be smooth, according to certain embodiments, and use a tension or friction fit to remain securely positioned in the female reproductive tract. Alternatively, the maximized outer surface area 228 may include a surface texture (not shown) configured to grip the desired area of the female reproductive tract and use a combination of surface texture, traction, and tension or friction fit to remain securely positioned in the female reproductive tract.

Any materials or combination of materials may be used to fabricate the support rings 242, as determined by one of skill in the art. As non-limiting examples, biosafe shape memory natural rubber, memory foam, plastic, and silicone may be used. Certain more particular embodiments may include support rings 242 including one or more coils (not shown) having a predetermined shape and capable of deformation during insertion and removal of the medical device 200. A skilled artisan may select any material or combination of materials that may include elastic or shape-memory properties capable of reverting the support rings 242 back to the predetermined shape after insertion or removal of the medical device 200, as desired. The support rings 242 may be fabricated using a strong, resilient material that is capable of withstanding pressure from the retracting vaginal walls of the patient.

The support rings 242 may be integral with the main body 204 of the medical device 200 or may be permanently connected or fastened to the main body 204 of the medical device 200. The support rings 242 may have any suitable shape, size, and configuration adapted to optimize the fluid tight seal and create tension between the medical device 200 and the vaginal wall or other area of the female reproductive tract.

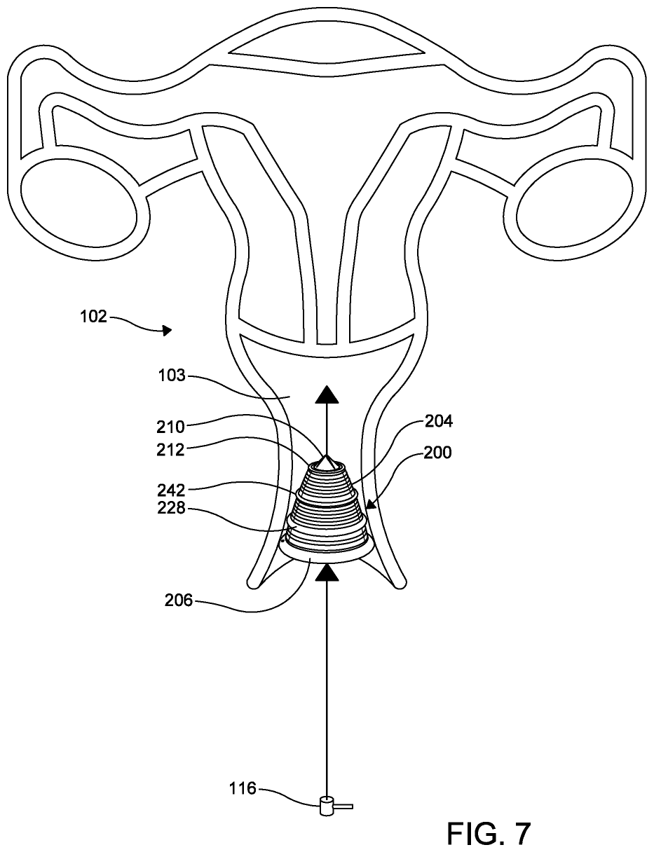

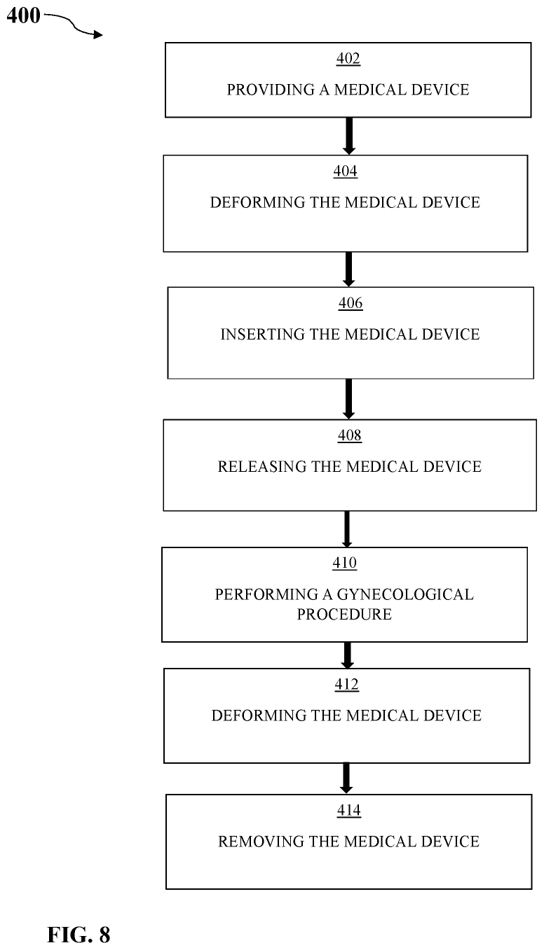

With reference now to FIGS. 1-7, it should be appreciated that the medical device 100, 200 may include more than one mode of operation depending on the orientation of the medical device 100, 200 in the patient. The medical device 100, 200 may be inserted into the patient leading with either the attachment rim 106, 206, as shown in FIGS. 1-6, or leading with the valve securing element 112, 212, as shown in FIG. 7. Where the medical device 100, 200 is inserted into the patient leading with the attachment rim 106, 206, the valve 110, 210 may extend upwardly from the valve securing element 112, 212, through the main body 104, 204 of the medical device 100, 200, and toward the attachment rim 106, 206. Alternatively, where the medical device 100, 200 is inserted leading with the valve securing element 112, 212, the valve 110, 210 may extend upwardly away from the main body 104, 204 and away from the valve securing element 112, 212 of the medical device 100, 200. In certain embodiments, the valve 110, 210 may be a flap valve 110, 210 capable of extending in both directions depending on the orientation of the medical device 100, 200 in the patient. In certain other embodiments, the valve 110, 210 may be a one-way valve 110, 210 in a set position and adapted to extend in only one direction. In both modes of operation, the attachment rim 106, 206 and, in certain embodiments, the support rings 242, are adapted to form a fluid tight seal with the vaginal walls or other areas of the female reproductive tract 102 of the patient.

In operation, the medical professional may sterilize the medical device 100 prior to use. After determining the appropriate mode of operation for the specific patient, the medical professional may deform the medical device 100 and begin inserting the medical device 100 into the patient's female reproductive tract 102. Once the medical device 100 is positioned in the desired location, the medical professional may release the medical device 100, thereby allowing the medical device 100 to resume the predetermined shape within the patient. Adjustments with respect to the fit within the patient, as well as the fluid tight seal between the medical device 100 and the patient's female reproductive tract 102 may be made as needed. According to certain embodiments, the medical professional may introduce fluid (e.g., air) into the attachment rim 106 in order to create a desired tension and a maximized outer surface area between the attachment rim 106 and the vaginal walls or other areas of the patient's female reproductive tract 102.

The medical professional may then insert an examination tool 116 through one or both of the first and second openings 120, 122 of the medical device 100 in order to perform a gynecological examination or procedure. Insertion of the examination tool 116 may properly position the valve 110 around the examination tool 116 to form a fluid tight seal. Upon completion of the gynecological examination or procedure, the medical professional may deform the medical device 100 and remove the medical device 100 from the patient.

Advantageously, the medical device 100 may be easily and atraumatically inserted and removed from the patient by deformation of the medical device 100. More specifically, the flexibility of at least the main body 104 and the attachment rim 106 allows the medical device 100 to be easily deformed for insertion and removal. The shape-memory properties of at least the main body 104 and the attachment rim 106 allow the medical device 100 to resume the predetermined shape 101 upon insertion in the patient. The absence of sharp edges or other protrusions, as well as the absence of small, loose parts, further minimizes risk of injury to the patient.

The attachment rim 106, having a maximized outer surface area 128 in contact with a desired area of the patient and providing optimal tension against a desired area of the patient forms a fluid tight seal with a desired area of the patient such as the vaginal wall, as one non-limiting example. Likewise, in certain embodiments, the adjustable nature of the attachment rim 106 and, in certain embodiments, the elastic properties of one or both the attachment rim 106 and the support rings 242 allow the medical device 100 to form a fluid tight seal in patients having variable anatomy. The flexible valve 110 allows for insertion of examination tools 116 having various diameters while providing an additional fluid tight seal. Accordingly, the medical device 100 militates against undesired fluid discharge. This may be especially important during examinations and procedures that require hydrodistension of the vaginal cavity, as one non-limiting example.

The valve securing element 112 having a semi-rigid or rigid configuration securely fastens the valve 110 to the main body 104 of the medical device 100 while also ensuring that the valve 110 is not altered or damaged during insertion or removal of the medical device 100. More specifically, the valve securing element 112 keeps the valve 110 in a desired configuration and position relative to the second opening 122 so that the fluid tight seal formed by the valve 110 is in place once the medical device 100 is properly positioned in the patient.

When the main body 104 includes a translucent material, the main body 104 can allows for direct visualization of the entire vagina of the patient including the vaginal walls and the first and second openings 120, 122 further improve direct visualization of the female reproductive tract 102 during and after insertion of the medical device 100.

More specifically, the medical device 100 allows for insertion of a hysteroscope, as one non-limiting example, and permits hydrodistension of the vagina and hydrodilation of the opening of the cervix under direct visualization, while simultaneously using a fluid tight seal formed by the attachment rim 106 and the valve 110 to prevent distention fluid from leaking out of the patient during the gynecological examination or procedure.

The flexible, adaptable medical device 100 may be used in patients of any age, as needed, in women with significant anteversion or retroversion of the uterus, and in women who have a stenotic cervical opening. Additionally, the medical device 100 is effective in multiparous or obese patients, and in patients with herniating pelvic organs. Additionally, the medical device 100 is easy to sterilize, may be disposable or reusable, as determined by one of skill in the art, and is easy and inexpensive to manufacture.

In yet another embodiment of the present disclosure, with reference to FIG. 8, a method 400 of using the medical device 100 to perform a gynecological procedure, is shown. The method 400 may include a step 402 of providing the medical device 100. The method 400 may further include another step 404 of deforming the medical device 100 prior to insertion into the female reproductive tract 102 of a patient. Another step 406 may include inserting the medical device 100 in a desired area of the female reproductive tract 102. In a next step 408, the medical device 100 may be released such that the medical device 100 may resume a predetermined shape within the female reproductive tract 102 of the patient. A next step 410 may include performing the gynecological procedure. Upon completion of the gynecological procedure, final steps 412, 414 may include deforming the medical device 100 and removing the medical device 100 from the female reproductive tract 102 of the patient.

It should be appreciated that the medical professional may need to perform additional steps, according to various embodiments of the present disclosure. As non-limiting examples, additional steps may include introducing fluid (e.g., air) into and removing fluid from the attachment rim 106 of the medical device 100, in order to inflate/deflate the attachment rim 106 as desired. As another non-limiting example, an additional step may include making adjustments to the location and the fit of the medical device 100 within the patient, as needed. It should be further appreciated that the medical professional may need to perform the steps in a different order or repeat steps, as needed. As one non-limiting example, the medical professional may introduce and remove various examination tools 116 multiple times during the gynecological examination or procedure.

Example embodiments are provided so that this disclosure will be through and fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, components and methods can be made within the scope of the present technology, with substantially similar results.

What is claimed is:

1. A medical device for use during a gynecological procedure, comprising:
a main body;
an attachment rim disposed at a first end of the main body;
a valve securing element disposed at a second end of the main body; and
a valve disposed between the main body and the valve securing element.

2. The medical device of claim 1, wherein the main body has a predetermined configuration that is substantially cup-shaped.

3. The medical device of claim 2, wherein the main body may be deformed when a pressure is applied thereto and wherein the main body resumes the predetermined configuration when the pressure is not applied thereto.

4. The medical device of claim 1, wherein the main body includes a translucent material.

5. The medical device of claim 1, wherein an outer surface of the main body includes a groove.

6. The medical device of claim 1, wherein the main body includes at least one flexible support ring disposed intermediate the first end and the second end.

7. The medical device of claim 1, wherein the attachment rim is configured to form a fluid tight seal with a vaginal wall of a patient.

8. The medical device of claim 1, wherein the attachment rim includes an expandable material and wherein the attachment rim is hollow.

9. The medical device of claim 8, wherein the attachment rim is in fluid communication with a tube, and wherein the tube is adapted to introduce a fluid into and remove air from the attachment rim.

10. The medical device of claim 1, wherein the valve securing element is rigid, and wherein the valve securing element has a ring-shaped configuration.

11. The medical device of claim 1, wherein the valve is one of integral with and permanently fastened to the valve securing element.

12. The medical device of claim 1, wherein the valve is a one-way valve.

13. The medical device of claim 1, wherein the valve is one of a flap valve, a duckbill valve, and a gel-port.

14. The medical device of claim 1, wherein the valve is adapted to receive examination tools having variable diameters and form a seal with the examination tools having variable diameters.

15. The medical device of claim 1, wherein the valve is one of:
configured to extend upwardly toward the attachment rim when the attachment rim is inserted into a patient first;
configured to extend upwardly away from the main body and the attachment rim when the valve securing element is inserted into the patient first; and
configured to extend upwardly toward the attachment rim and upwardly away from the main body and the attachment rim, depending on an orientation of the medical device upon insertion into the patient.

16. A medical device for use during a gynecological procedure, comprising:
a main body;
an attachment rim disposed at a first end of the main body;
a valve securing element disposed at a second end of the main body;
a first valve disposed between the main body and the valve securing element; and
a second valve disposed between the main body and the valve securing element.

17. The medical device of claim 16, wherein at least one of the first valve and the second valve is a flap valve.

18. The medical device of claim 16, wherein the first valve is a tri-leaflet valve and the second valve is a bi-leaflet valve.

19. A method of using a medical device during a gynecological procedure, comprising:
providing the medical device for use during the gynecological procedure, the medical device including a main body, an attachment rim disposed at a first end of the main body, a valve securing element disposed at a second end of the main body, and a valve disposed between the main body and the valve securing element;
inserting the medical device into a patient;

20. The method of claim 19, further comprising the steps of:
deforming the medical device prior to inserting the medical device into a patient;
releasing the medical device in the patient;
performing a gynecological procedure;
deforming the medical device; and
removing the medical device from the patient.
